(19) United States
(12) Patent Application Publication
(10) Pub. No.: US 2010/0285163 A1
(43) Pub. Date: Nov. 11, 2010

(54) Composition for Treating or Preventing Hot Flashes and use Thereof

(76) Inventor: Ping Ping Li, Beijing (CN)

Correspondence Address: MARSHALL, GERSTEIN & BORUN LLP, 233 SOUTH WACKER DRIVE, 6300 WILLIS TOWER, CHICAGO, IL, 60606-6357, US

(21) Appl. No.: 12/160,772

(22) PCT Filed: Jan. 12, 2007
(86) PCT No.: PCT/SG07/00010

§ 371 Date: Feb. 17, 2009

(30) Foreign Application Priority Data
Jan. 12, 2006 (SG) 200600216-6

(51) Int. Cl. A61K 36/00; A61P 5/24

(52) U.S. Cl. 424/773

(57) ABSTRACT

The present invention provides compositions and methods of their use for treating or preventing climacteric symptoms such as hot flashes. A composition of the invention preferably comprises Chaihu, Yujin, Mudanpi, Zicao, Baiwei, Baishao and Wuweizi or extracts thereof, and is preferably administered as a single composition.

### FIELD OF THE INVENTION

The invention relates to a composition and method for treating or preventing climacteric symptoms such as hot flashes. More particularly, the invention relates to use of Traditional Chinese Medicine (TCM) to treat or prevent hot flashes.

### BACKGROUND TO THE INVENTION

Climacteric symptoms are related to endocrine, somatic and psychological changes that occur at the termination of the reproductive period in women. Climacteric symptoms include the occurrence of hot flashes, which may in turn impair sleep quality.

The occurrence of hot flashes in women can cause significant discomfort and pain. Whilst hot flashes are more commonly associated with natural menopause, they can also be caused by drug-induced menopause or surgical-induced menopause.

Natural menopause is due to the natural physiological senescence of ovaries that every woman experiences as she ages.

Drug-induced menopause could refer to patients receiving treatment for a gynaecological cancer (breast, endometrial, cervical, ovarian or vaginal) or for any gynaecological disease for which estrogen and progesterone secretion must be blocked to stop the disease's progression.

Gynaecological cancers such as breast, ovary, endometrial or vaginal cancers can be estrogen-sensitive (estrogen receptors are present in the tumor cells) or non-estrogen sensitive. For the estrogen sensitive cancers, the usual therapy (also called chemotherapy) is a hormonotherapy (also known asendocrine therapy) alone or in combination with other anti-cancer treatments (other chemotherapy, radiation, surgery, etc). For non-estrogen sensitive cancers, the usual therapy does not involve hormonotherapy but instead relies on typical anti-cancer treatments (chemotherapy, radiation, surgery, etc). Typically, drug-induced menopause, and thus hot flashes are induced only by the chemotherapy given for estrogen sensitive cancers, also defined as hormonotherapy.

Such hormonotherapy is also used in others gynaecological disorders such as endometriosis for which estrogen and progesterone secretion must be blocked to stop the disease' progression.

Thus, ‘drugs’ inducing menopause could be referred to as chemotherapy, anti-cancer therapy, immunotherapy, hormonotherapy or endocrine therapy.

Surgical-induced menopause refers to menopause caused by hysterectomy (surgical removal of the uterus) performed for various gynaecological reasons. These include intolerable periods not controlled by medical means, fibroids, endometriosis, prolapse, and malignant or premalignant changes of the uterus, cervix or ovary. Hysterectomy can either be total (where both the uterus and cervix are removed), or sub-total (where the main part of the uterus is removed). Ovaries may be conserved (left behind) or removed. If one or both ovaries are conserved, they may continue producing hormones in their fluctuating manner until the normal age of menopause, or they may stop producing hormones sooner than expected, inducing an early menopause. This can even happen within 1-2 years following the hysterectomy when symptoms of oestrogen deficiency may be noticed.

The main, and only treatment to date for hot flashes is the use of hormonotherapy such as estrogens alone, estrogens combined with progesterone, or phytoestrogens. However, the use of estrogens as Hormone Replacement Therapy (HRT) has been shown by the World Health Organisation (WHO) to increase the risk of gynaecological cancers. Whilst phytoestrogens have not yet been tested, they are also suspected of causing a similar increased risk of cancer as they are believed to bind the same estrogen-receptors.

The current lack of understanding of hot flashes is reflected both in the lack of consensus over its prevalence in women, and also by the multitude of theories seeking to explain its aetiology. The most widely accepted theory is that hot flashes are caused by a deficiency in circulating hormones (estrogen and progesterone) as a result of declining or disturbed ovarian function. It is known that fluctuating hormone levels are at least part of the picture, because estrogen replacement therapy relieves the symptoms of hot flashes in many women.

However, this theory does not fully explain the phenomenon of hot flashes. Many menopausal and pre-menopausal women with low estrogen levels never experience hot flashes, while other women simultaneously experience hot flashes and symptoms of a relative estrogen excess. Signs of estrogen excess or estrogen dominance include weight gain, breast tenderness, heavy menstrual flow and erratic mood swings.

Hot flashes are a complex neuro-endocrine phenomenon affected by a variety of factors including our thoughts, expectations and emotions. The wide range of stimuli that trigger flushing further attests to the multi-factorial nature of hot flashes. Common triggers include: spicy food, hot drinks, alcohol, sugar, caffeine, stress, hot weather, hot tubs and saunas, tobacco and “heated” emotions.

Another explanation for hot flashes is that they are triggered by a brief but sudden downward adjustment in the body's internal temperature setting. It is known that both estrogen and progesterone play a role in temperature regulation. However, the role of these two hormones, or a multitude of others, in triggering a shift in the body's thermoregulatory centre is not known. Some research indicates that hot flashes may be triggered when declining levels of estrogen and progesterone cause a withdrawal of naturally occurring opiates (chemicals in the brain that have a significant impact on mood, pain control and hormone modulation).

Studies indicate that stabilization of either estrogen or progesterone can minimize hot flashes but data from a recent study (refer to the National Institutes of Health finding from their Woman's Health Initiative about the benefits and risks of menopausal hormone therapy) using estrogen plus progestin (also known as progesterone) therapy in menopausal women have shown that these treatments probably increase the risk of gynaecological cancers.

A number of medicinal plants used for centuries by indigenous cultures have gained reputations as female tonics. The tonifying effects of these herbs are most likely due to the presence of phytoestrogens, as well as the plants' abilities to nourish the endocrine glands and increase blood flow to the pelvic organs. Herbs commonly used to alleviate hot flashes include black cohosh, motherwort, chaste tree berry, blue cohosh, ginseng, dong quai, liquorice, sarsaparilla and false unicorn but the effect of these phytoestrogens on gynaecological cancers has not yet been investigated.

Another area in which hot flashes are seen is following chemotherapy, anti-cancer therapy, immunotherapy, hormonotherapy or endocrine therapy given to treat breast cancer patients. Breast cancer is the most common malignant tumour in women. The use of chemotherapy, anti-cancer therapy, immunotherapy, hormonotherapy or endocrine therapy given targeting gynaecological organs, leads to various menopausal side-effects such as climacteric symptoms which includes hot flashes due to the very low level of circulating hormones. The persistent hot flashes due to drug-induced menopause can last for 2 to 5 years and impair the quality of life in gynaecological cancer patients greatly.

Thus, the treatment/regressions of hot flashes is one of the focuses to improve the quality of life in gynaecological cancer patients.

As for breast cancer patients, the treatment of hot flashes occurring in other gynaecological cancers is a difficult problem and as yet there is still no effective method of control as Hormone Replacement Therapy which is the only available treatment. Hormone Replacement Therapy is however totally contra-indicated because it could stimulate the growth and multiplication of breast cancer cells. Some selective 5-TH re-absorption inhibitors can improve hot flashes but the latest studies show that they may reduce the therapeutic effect of hormonotherapeutic agents such as tamoxifen (TAM).

There is an invaluable wealth of knowledge that has been developed through the use of Traditional Chinese Medicine in the last approximately 4000 years.

The use of compositions comprising a large variety of herbs are known for the treatment of hormonal imbalances including menopausal syndrome, menstrual irregularities, regulation of climacteric syndrome and the treatment of cancer.

However, these compositions generally involve the use of a large number of herbs. For example CN1321473 describes the use of an 11 component mixture for curing menopausal syndrome, CN1440776 describes the use of a 23 component mixture for regulating menstruation, U.S. Pat. No. 6,238,707 describes the use of a 26 component mixture that provides benefits for female hormonal regulation or replacement and EP1462110 describes the use of an 18 component mixture for the treatment of hormonal symptoms. In addition, these preparations all contain at least one of the following phytoestrogens
black cohosh (Cimicifuga racemosa)
dong quai (Angelica sinensis) or Dang Gui
red clover (Trifolium pratense)
alfalfa (Medicago sativa)
licorice (Glycyrrhiza glabra)
Korean ginseng (Panax ginseng)
wild American ginseng (Panax quinquefolius)
Kudzu root (Puerariae lobata).

The effects of these phytoestrogens on gynaecological cancers have not yet been investigated.

It should be appreciated that the use of herbal compositions can have unwanted side-effects. These side-effects are exacerbated by the use of large preparations containing a large number of herbs which increase the likelihood that certain unwanted effects will also be passed on to the patient.

As discussed above, it has recently been determined that the use of HRT provides a health risk to those woman receiving it. Such increased risks include breast cancer (Warren et al., “Strategies for managing breast cancer risk after the menopause.” Treatments in Endocrinology 2004; 3(5):289-307; Diamanti-Kandarakis E. “Hormone replacement therapy and risk of malignancy.” Current Opinion in Obstetrics & Gynecology 2004; 16(1):73-8; Timins J. K. “Current issues in hormone replacement therapy.” New Jersey Medicine 2004; 101(3):21-7)

Thus, there is a significant advantage in being able to administer a medicament that is able to ameliorate menopausal symptoms without the side-effects of increased cancer risks. In addition, the same advantage is seen when considering gynaecological cancer-patients for which HRT is highly contra-indicated as it causes an increased risk of cancer cell growth and cancer relapse.

It would be an advantage of the present invention if embodiments provide a composition that does not contain estrogens or known phytoestrogens and that can ameliorate, treat or prevent climacteric symptoms such as hot flashes, or to at least provide the public with a useful choice.

Other advantages associated with embodiments of the invention may become apparent from the following description which is given by way of example only.

### SUMMARY OF THE INVENTION

One aspect provides a composition comprising Wuweizi or an extract thereof, and Baishao or an extract thereof, in combination with at least one of Baiwei or an extract thereof, Zicao or an extract thereof, Mudanpi or an extract thereof. Yujin or an extract thereof, or Chaihu or an extract thereof.

Another aspect provides a composition comprising Chaihu, Yujin, Mudanpi, Zicao, Baiwei, Baishao and Wuweizi or extracts thereof.

Preferably, the composition is a pharmaceutical composition further comprising a pharmaceutically acceptable carrier.

Preferred extracts are water extracts.

In one embodiment, a composition comprises one or more hormonotherapeutic agents. The hormonotherapeutic agent may be tamoxifen (TAM).

In one embodiment, the composition is administered simultaneously or sequentially with chemotherapeutic, anti-cancer, immunotherapeutic, hormonotherapeutic or endocrine therapeutic agents used for gynaecological cancers such as tamoxifen, taxanes, taxol, paclitaxel (taxol), doxetaxel (taxotere), cyclophosphamide (docettaxel), alkylating agents (mitotic inhibitors), doxorubicin (paditaxel, adriamycin), antibiotics, mitoxantrone (novantrone), vinorelbine (navelbine), vinca alakaloids (mitotic inhibitors), fluoro-uracile, pyrimidine analogs (antimetabolites), capecitabine (xeloda), methotrexate, folic acid analog (anti-metabolites); hormonal therapies such as tamoxifen (Nolvadex)—antiestrogens, toremifene (fareston)—antiestrogens, letrozole (Femara)—aromatase inhibitors, anastrazole (Arimidex)—aromatase inhibitors, exemestane (Aromasin)—aromatase inhibitors, goserelin (Zoladex)—LHRH inhibitors, leuprolide (Leurpon)—LHRH inhibitors, fulvestrant (Faslodex); surgery (eg, lumpectomy or mastectomy for breast cancers); radiation; monoclonal antibody therapy (trastuzumab (herceptin).

Another aspect of the invention provides a method of reducing the frequency and/or severity of climacteric symptoms in a subject, the method comprising administering to a subject in need thereof, a composition comprising Wuweizi or an extract thereof, and Baishao or an extract thereof, in combination with at least one of Baiwei or an extract thereof, Zicao or an extract thereof, Mudanpi or an extract thereof, Yujin or an extract thereof, or Chaihu or an extract thereof.

Another aspect provides a method of reducing the frequency and/or severity of symptoms caused by drug-induced menopause in a subject, the method comprising administering to a subject in need thereof a composition comprising Wuweizi or an extract thereof, and Baishao or an extract thereof, in combination with at least one of Baiwei or an extract thereof, Zicao or an extract thereof, Mudanpi or an extract thereof, Yujin or an extract thereof, or Chaihu or an extract thereof. Drug-induced menopause includes menopause induced by chemotherapy, anti-cancer therapy, immunotherapy, hormonotherapy and endocrine therapy.

Another aspect provides a method of reducing the frequency and/or severity of symptoms caused by surgical-induced menopause in a patient, the method comprising administering to a subject in need thereof a composition comprising Wuweizi or an extract thereof, and Baishao or an extract thereof, in combination with at least one of Baiwei or an extract thereof, Zicao or an extract thereof, Mudanpi or an extract thereof, Yujin or an extract thereof, or Chaihu or an extract thereof. Surgical-induced menopause includes menopause induced by hysterectomy. Another aspect provides a method of reducing the frequency and/or severity of climacteric symptoms such as hot flashes in natural pre-menopausal and menopausal women, the method comprising administering to a subject in need thereof a composition comprising Wuweizi or an extract thereof, and Baishao or an extract thereof, in combination with at least one of Baiwei or an extract thereof, Zicao or an extract thereof, Mudanpi or an extract thereof, Yujin or an extract thereof, or Chaihu or an extract thereof.

Another aspect provides a method of reducing the frequency and/or severity of climacteric symptoms such as hot flashes in patients with a gynaecological cancer, the method comprising administering to a subject in need thereof a composition comprising Wuweizi or an extract thereof, and Baishao or an extract thereof, in combination with at least one of Baiwei or an extract thereof, Zicao or an extract thereof, Mudanpi or an extract thereof, Yujin or an extract thereof, or Chaihu or an extract thereof.

In one embodiment, the subjects are those for which Hormone Replacement Therapy (HRT) would increase the risk of gynaecological cancer onset or relapse.

Other aspects provide use of a composition comprising Wuweizi or an extract thereof, and Baishao or an extract thereof, in combination with at least one of Baiwei or an extract thereof, Zicao or an extract thereof, Mudanpi or an extract thereof, Yujin or an extract thereof, or Chaihu or an extract thereof, in the preparation of a medicament for reducing the frequency and/or severity of climacteric symptoms such as hot flashes, reducing the frequency and/or severity of symptoms before or during natural menopause, reducing the frequency and/or severity of symptoms caused by drug-induced (chemotherapy, anti-cancer therapy, immunotherapy, hormonotherapy or endocrine therapy) menopause symptoms in patients with a gynaecological cancer or in patients with gynaecological disorders that require chemotherapy, anti-cancer therapy, immunotherapy, hormonotherapy or endocrine therapy, or reducing the frequency and/or severity of symptoms caused by surgical-induced menopause symptoms in patients with hysterectomy. Preferred extracts are water based extracts substantially comprising water. In one embodiment, the medicament further comprises a pharmaceutically acceptable carrier.

In one embodiment, the subjects are administered said medicament simultaneously or sequentially with one or more hormonotherapeutic agents. Preferably, the hormonotherapeutic agent is tamoxifen (TAM).

In one embodiment, said medicament is administered as a single composition.

Another aspect provides a process for preparing a composition comprising the step of obtaining a herbal extract from a solvent containing Wuweizi or an extract thereof, and Baishao or an extract thereof, in combination with at least one of Baiwei or an extract thereof, Zicao or an extract thereof, Mudanpi or an extract thereof, Yujin or an extract thereof, or Chaihu or an extract thereof. Another aspect provides a method of preparing a composition comprising:

1. placing a mixture of Chaihu, Yujin, Mudanpi, Zicao, Baiwei, Baishao and Wuweizi into a solvent,
2. heating the mixture until boiling,
3. simmering the mixture to enrich the solvent, and
4. collecting the enriched solvent.

In one embodiment, the solvent is alcohol or water or a mixture thereof.

In one embodiment, the solvent is water.

In one embodiment, the method can be repeated multiple times by adding a quantity of water to the composition that is approximately equal to the amount of enriched water that is removed.

Preferably, the enriched water is administered to a subject in need thereof.

Preferably, about 100 ml of enriched water is administered per day. More preferably, about 100 ml of enriched water is administered at least two times per day.

In one embodiment, the enriched water is freeze or spray dried. Preferably, the dried enriched water is included in a suitable dosage form. Preferably, the dosage form is administered at least 1, 2, 3, 4 or 5 times per day. Most preferably the dosage form is administered 2 times per day.

The following preferred embodiments may relate to any of the above aspects.

Preferably Chaihu, Yujin, Mudanpi, Zicao, Baiwei, Baishao and Wuweizi or extracts thereof are administered as a single composition.

In one embodiment, the composition comprises:

(a) about 3% (dry weight) to about 12% (dry weight) of Chaihu or an extract thereof,
(b) about 3% (dry weight) to about 12% (dry weight) of Yujin or an extract thereof,
(c) about 5% (dry weight) to about 15% (dry weight) of Mudanpi or an extract thereof,
(d) about 12% (dry weight) to about 20% (dry weight) of Zicao or an extract thereof,
(e) about 5% (dry weight) to about 15% (dry weight) of Baiwei or an extract thereof,
(f) about 5% (dry weight) to about 15% (dry weight) of Baishao or an extract thereof, and
(g) about 5% (dry weight) to about 15% (dry weight) of Wuweizi or an extract thereof, or multiples thereof yet retaining approximate proportionality.

Preferably, the composition comprises about 10% (dry weight) of Chaihu, about 10% (dry weight) of Yujin, about 15% (dry weight) of Mudanpi, about 20% (dry weight) of Zicao, about 15% (dry weight) of Baiwei, about 15% (dry weight) of Baishao and about 15% (dry weight) of Wuweizi or multiples thereof yet retaining approximate proportionality.

Preferred compositions of the invention comprise about 1 to about 30 g of each component. More preferred compositions of the invention comprise:

(a) about 5, about 6, about 7, about 8, about 9, about 10, about 11, about 12, about 13, about 14 or about 15 g of Chaihu or an extract thereof,
(b) about 5, about 6, about 7, about 8, about 9, about 10, about 11, about 12, about 13, about 14 or about 15 g of Yujin or an extract thereof,
(c) about 10, about 11, about 12, about 13, about 14, about 15, about 16, about 17, about 18, about 19 or about 20 g of Mudanpi or an extract thereof,
(d) about 15, about 16, about 17, about 18, about 19, about 20, about 21, about 22, about 23, about 24 or about 25 g of Zicao or an extract thereof,
(e) about 10, about 11, about 12, about 13, about 14, about 15, about 16, about 17, about 18, about 19 or about 20 g of Baiwei or an extract thereof,
(f) about 10, about 11, about 12, about 13, about 14, about 15, about 16, about 17, about 18, about 19 or about 20 g of Baishao or an extract thereof, and
(g) about 10, about 11, about 12, about 13, about 14, about 15, about 16, about 17, about 18, about 19 or about 20 g of Wuweizi or an extract thereof,
or multiples thereof yet retaining approximate proportionality, and useful ranges may be selected between any of these forgoing values (e.g. from about 8 to about 10 g).

Most preferably, the composition comprises about 10 g of Chaihu, about 10 g of Yujin, about 15 g of Mudanpi, about 20 g of Zicao, about 15 g of Baiwei, about 15 g of Baishao and about 15 g of Wuweizi or multiples thereof yet retaining approximate proportionality.

In one embodiment, the composition comprises plant material. Said plant material may be in a dried or fresh form.

In one embodiment, the composition comprises extracts, preferably water extracts, of plant material.

In one embodiment, the plant material is mixed with water and soaked for 30 minutes with the water level higher than the composition level. Preferably the water level is approximately about 1, about 2, about 3, about 4, about 5, about 6, about 7, about 8, about 9, or about 10 cm higher than the composition level.

Alternatively, the amount of water added to the plant material can be based on the amount of plant material used. In one embodiment, about 50, about 100, about 200, about 300, about 400, about 500, about 600, about 700, about 800, about 900, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700 or about 3000 ml of water is added per about 100 g of dried plant material. Preferably, about 300 to about 3000 ml, more preferably about 1000 ml of water is added per about 100 g of dried herb.

Preferably, the mixture is heated to boiling and simmered for about 10, about 15, about 20, about 25, about 30, about 35, or about 40 minutes. More preferably, the mixture is simmered for about 15 to about 25 minutes, most preferably about 20 minutes.

Unless specified otherwise, the terms “comprising” and “comprise”, and grammatical variants thereof, are intended to represent “open” or “inclusive” language such that they include recited elements but also permit inclusion of additional, unrecited elements.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value. Other aspects may become apparent from the following description which is given by way of example only.

### DETAILED DESCRIPTION OF THE INVENTION

The disclosed embodiments provide compositions and methods which are useful to treat or prevent climacteric symptoms such as hot flashes, whether they arise spontaneously following natural menopause or they are cause by drug-induced (chemotherapy, anti-cancer therapy, immunotherapy, hormonotherapy or endocrine therapy) menopause or surgical-induced (hysterectomy) menopause.

Natural menopause is due to the natural physiological senescence of ovaries that every woman experiences as she ages.

Drug-induced menopause refers to patients receiving treatment for a gynaecological cancer (breast, endometrial, ovarian, cervical or vaginal) or for any gynaecological disease for which estrogen and progesterone secretion must be blocked to stop the disease's progression.

Surgical-induced menopause refers to menopause caused by hysterectomy (surgical removal of the uterus) performed for various gynaecological reasons.

Particularly, the compositions and methods are useful to reduce the frequency and/or severity of hot flashes. Preferably, the frequency and/or severity of hot flashes is reduced. Preferably, hot flashes cease.

The compositions and methods disclosed herein are useful to treat or prevent climacteric symptoms such as hot flashes resulting from natural menopause, drug-induced (chemotherapy, anti-cancer therapy, immunotherapy, hormonotherapy or endocrine therapy) menopause or (hysterectomy) surgical-induced menopause.

The compositions disclosed herein generally comprise Wuweizi or an extract thereof, and Baishao or an extract thereof, in combination with at least one of Baiwei or an extract thereof, Zicao or an extract thereof, Mudanpi or an extract thereof, Yujin or an extract thereof, or Chaihu or an extract thereof.

The method disclosed herein generally comprises preparing a decoction from herbs and then administering to a patient or subject in need thereof.

As used herein the term “hot flashes” refers to the intense heat sensation, flushing, profuse sweating, palpitations, and/or sense of anxiety experienced by subjects undergoing natural menopause, drug-induced (chemotherapy, anti-cancer therapy, immunotherapy, hormonotherapy or endocrine therapy) menopause or surgical-induced (hysterectomy) menopause. Hot flashes can also occur independent of any such therapy or condition. Hot flashes impact on a subject's quality of life, including their ability to sleep.

Chaihu, Yujin, Mudanpi, Zicao, Baiwei, Baishao and Wuweizi are all herbs commonly used in Traditional Chinese Medicine.

Chaihu is commonly known as hares ear root or Chinese thorowax root. Its Latin name is Radix Bupleuri and the botanical species are known as Bupleurum chinense DC (Northern Chinese Thorowax Root) and Bupleurum scorzonerifolium Willd. (Southern Chinese Thorowax Root).

Yujin is commonly known as turmeric root tuber. Its Latin name is Radix Curcumae and its botanical names are Curcuma wenyujin, Curcuma longa or Curcuma phaeocaulis Val.

Mudanpi is commonly known as tree peony bark and is the dried root bark of Paeonia suffruticosa andr. Its Latin name is Cortex Moutan.

Zicao is commonly known as Arnebia root or gromwell root. Its Latin name is Radix Arnebiae and its botanical names are Arnebia euchroma (Royle) Johnst., Lithospermum erythrorhizon Sieb. Et Zucc., or Arnebia guttata Bunge.

Baiwei is commonly known as blackened swallowwort root. Its Latin is Radix Cynanchi Atrati and botanical names are Cynanchum atratum Bge. or Cyanchum versicolor Bge.

Baishao is commonly known as white peony root, and is known by the Latin and botanical names of Radix Paeoniae Alba and Paeonia lactiflora Pall. respectively.

Wuweizi is commonly known as Chinese Magnoliavine fruit and is known by the Latin and botanical names of Frutus Schisandrae Chinensis and Schisandra chinensis (Turcz.) Barll. (northern Wuweizi) or Schisandra sphenanthera Rehd. (southern Wuweizi) respectively.

Each of Chaihu, Yujin, Mudanpi, Zicao, Baiwei, Baishao and Wuweizi are commercially available and referred to in common Traditional Chinese Medicine texts.

As used herein the term “treat” includes the prevention, amelioration or reduction in severity or frequency of any one or more of the symptoms associated with hot flashes. The term “treat” and its derivatives should be interpreted in their broadest possible context. The term should not be taken to imply that a subject is treated until total recovery.

As used herein the term “and/or” means “and” or “or” or both.

As used herein the term “extract” includes a concentrate of a herb. It will be appreciated that either the herb itself or an extract thereof may be useful for use in medicine. The extract may be obtained by known art methods including, but not limited to, infusion, decoction, macero-expression, percolation, expression, distillation and supercritical extraction. Said processes may use mixtures such as solvents to aid in the extraction of the soluble components of the herb. The mixtures used to aid extraction include those solutions known in the art to be useful in such extraction processes. Preferably said solvents are selected from an alcohol, organic solvent, halogenated solvent or water or a mix thereof. Useful solvents for supercritical extraction include compressed carbon dioxide. Reference to an herb herein is also intended to include an extract derived from that herb. A skilled worker with regard to that skill and the teaching of the present specification will readily be able to prepare an extract of a herb useful herein.

It should be understood that combinations of extracts or combinations of fresh or dried herbs and extracts may be used in a composition of the invention.

The present invention also provides a process for preparing a composition. The process generally comprises:

(a) providing a mixture comprising Wuweizi or an extract thereof, and Baishao or an extract thereof, in combination with at least one of Baiwei or an extract thereof, Zicao or an extract thereof, Mudanpi or an extract thereof, Yujin or an extract thereof, or Chaihu or an extract thereof,
(b) extracting the mixture with a solvent,
(c) collecting the extract-rich solvent; and
(d) concentrating said extract-rich solvent.

In one embodiment, about 50, about 100, about 200, about 300, about 400, about 500, about 600, about 700, about 800, about 900 or about 1000 ml of solvent is added per about 100 g of plant material.

Step (b) of the process of the invention may be carried out using one of infusion, decoction, macero-expression, percolation, expression, distillation or supercritical extraction.

Step (d) of the invention may be carried out using one of distillation, evaporation or percolation.

The extraction process may be a batch process, a semi-continuous process or a continuous process. In a batch process, the herb and the solvent may simply be added to a batch reactor, preferably one equipped with heating and stirring elements, and the extraction allowed to take place. In a semi-continuous or continuous mode of operation, a reactor comprising a series of extraction or percolation columns may be used.

In one embodiment, the reactor in which the extraction process occurs may be coupled to an apparatus for concentration and drying processes. Preferred concentration and drying processes include filtration, distillation, evaporation (including heat and or pressure evaporation), freeze drying and spray drying.

In one embodiment, the use of a maceration procedure comprises soaking the herb or herbs in a solution such as alcohol or water or a mixture thereof prior to filtering.

In an alternate embodiment, a counter-current process may be used for extraction. In this process a herb or herbs enter into a column of a large percolator comprised of several smaller columns. Material to be extracted is pumped through the different columns at a given temperature and flow speed where it continuously mixes with solvent. The extract-rich solvent then passes into another column, while fresh solvent once again comes into contact with herbal material as it is passed into a new chamber. The extract rich solvent can then be concentrated by known art methods such as distillation, evaporation or percolation.

In one embodiment, the extraction process may use a supercritical process of extraction. Preferably a gas is pressurised and heated to its supercritical temperature such that it remains a gas. Preferably said gas is carbon dioxide (CO2) although it should be appreciated that any suitable gas could be used.

The composition is useful in methods of reducing the frequency and/or severity of climacteric symptoms such as hot flashes following natural menopause.

The disclosed composition is useful in methods of reducing the frequency an/or severity of climacteric symptoms such as hot flashes caused by drug-induced (chemotherapy, anti-cancer therapy, immunotherapy, hormonotherapy or endocrine therapy) menopause when such drug are given to treat gynaecological (breast, ovarian, endometrial, cervical, vaginal) cancer patients or to improve patients with gynaecological disorders such as endometriosis.

The disclosed composition is useful in methods of reducing the frequency and/or severity of climacteric symptoms such as hot flashes caused by surgery (hysterectomy).

Chaihu, Yujin, Mudanpi, Zicao, Baiwei, Baishao and Wuweizi or extracts thereof may also be used in the preparation of a medicament for reducing the frequency and/or severity of hot flashes, reducing the frequency and/or severity of climacteric symptoms such as hot flashes, reducing the frequency and/or severity of symptoms before or during natural menopause, reducing the frequency and/or severity of symptoms caused by drug-induced (chemotherapy, anti-cancer therapy, immunotherapy, hormonotherapy or endocrine therapy) menopause symptoms in patients with gynaecological cancer or in patients with gynaecological disorders that require such treatment, reducing the frequency and/or severity of symptoms caused by surgical-induced menopause in patients who underwent hysterectomy.

Preferred compositions disclosed herein comprise 1 to 30 g of each component. More preferred compositions disclosed herein comprise:

(a) 5, 6, 7, 8, 9, 10, 11, 12, 13, 14 or 15 g of Chaihu or an extract thereof,
(b) 5, 6, 7, 8, 9, 10, 11, 12, 13, 14 or 15 g of Yujin or an extract thereof,
(c) 10, 11, 12, 13, 14, 15, 16, 17, 18, 19 or 20 g of Mudanpi or an extract thereof,
(d) 15, 16, 17, 18, 19, 20, 21, 22, 23, 24 or 25 g of Zicao or an extract thereof,
(e) 10, 11, 12, 13, 14, 15, 16, 17, 18, 19 or 20 g of Baiwei or an extract thereof,
(f) 10, 11, 12, 13, 14, 15, 16, 17, 18, 19 or 20 g of Baishao or an extract thereof, and
(g) 10, 11, 12, 13, 14, 15, 16, 17, 18, 19 or 20 g of Wuweizi or an extract thereof,
or multiples thereof yet retaining approximate proportionality, and useful ranges may be selected between any of these forgoing values (e.g. from about 8 to about 10 g), or multiples thereof yet retaining approximate proportionality, and useful ranges may be selected between any of these forgoing values (e.g. from about 10 to about 15 g).

Most preferably the composition comprises approximately 10 g of Chaihu, 10 g of Yujin, 15 g of Mudanpi, 20 g of Zicao, 15 g of Baiwei, 15 g of Baishao and 15 g of Wuweizi or multiples thereof yet retaining approximate proportionality.

It should be appreciated that larger amounts of the herbs could be prepared. However, the relative amounts of each herb to one another should still remain proportionally approximately the same.

In one embodiment the composition comprises plant material. Said plant material may be in a dried or fresh form.

In one embodiment the composition comprises extracts, preferably water extracts, of plant material.

A preferred method of preparing a composition of the invention comprises mixing appropriate amounts of the herbs together and decocting the herbal composition in alcohol or water or a mixture thereof.

Preferably the amount of alcohol or water or a mixture thereof that is used is approximately 1, 2, 3, 4, 5, 6 7, 8, 9, or 10 cm above the herbal composition. Most preferably the alcohol or water or a mixture thereof level is 5 cm above the herbal composition.

Alternatively, the amount of alcohol or water or a mixture thereof added to the plant material can be based on the amount of plant material used. In one embodiment approximately 50, 100, 200, 300, 400, 500, 600, 700, 800, 900 or 1000 ml of alcohol or water or a mixture thereof is added per 100 g of dried plant material. Preferably 500 ml of alcohol or water or a mixture thereof is added per 100 g of dried herb.

Once the alcohol or water or a mixture thereof and herbal composition have been combined in a container the herbal composition is allowed to soak in said alcohol or water or a mixture thereof for approximately 10, 15, 20, 25, 30, 35, or 40 minutes. More preferably the herbal composition is allowed to soak for 30 minutes. Following this the mixture is then heated and, once heated to a boil, is simmered for approximately 10, 15, 20, 25, 30, 35, or 40 minutes. More preferably the mixture is heated for 20 to 30 minutes. Preferably said container is non-aluminium.

Following this, an aliquot of the enriched alcohol or water or a mixture thereof is removed. Preferably the quantity of enriched alcohol or water or a mixture thereof removed is 70, 80, 90, 100, 110, 120 or 130 ml. More preferably the quantity of enriched alcohol or water or a mixture thereof removed is 100 ml.

Another aspect provides methods of preparing a composition comprising:

1. heating a herbal mixture of Chaihu, Yujin, Mudanpi, Zicao, Baiwei, Baishao and Wuweizi in a solvent to obtain an extract of said herbs, and
2. enriching said herbal extract by at least partly removing said solvent.

Preferably the herbs are placed in 2, 3, 4, 5, 6, 7, 8, 9, or 10 cm of alcohol or water or a mixture thereof. More preferably the herbs are placed in 5 cm of alcohol or water or a mixture thereof. Alternatively, the amount of alcohol or water or a mixture thereof added to the plant material can be based on the amount of plant material used. In one embodiment approximately 50, 100, 200, 300, 400, 500, 600, 700, 800, 900 or 1000 ml of alcohol or water or a mixture thereof is added per 30 g of dried herb. Preferably 500 ml of alcohol or water or a mixture thereof is added per 100 g of dried herb.

Preferably the herbs are soaked in the alcohol or water or a mixture thereof for 10, 20, 30 or 40 minutes before heating.

Preferably the mixture is heated for 10 to 40 minutes. Preferably the mixture is heated until boiling.

Preferably, the mixture is simmered for 10 to 40 minutes.

In one embodiment, the method can be repeated multiple times by adding a quantity of alcohol or water or a mixture thereof to the composition that is approximately equal to the amount of enriched alcohol or water or a mixture thereof that is removed.

Preferably, the amount of enriched alcohol or water or a mixture thereof that is removed at the end of each cycle is 70, 80, 90, 100, 110, 120 or 130 ml. More preferably the amount of enriched alcohol or water or a mixture thereof that is removed at the end of each cycle is 100 ml.

Once removed the enriched alcohol or water or a mixture thereof can be administered to a patient in need thereof. Preferably about 100 ml of enriched alcohol or water or a mixture thereof is administered per day. More preferably about administered 100 ml of the enriched alcohol or water or a mixture thereof is administered at least two times per day.

In one embodiment, the enriched alcohol or water or a mixture thereof is further processed to produce a suitably dosage form, such as a granule, tablet or capsule containing the enriched alcohol or water or a mixture thereof in liquid or dried from. Preferably the granule, tablet or capsule is administered to the patent or subject at least 1, 2, 3, 4 or 5 times per day. Most preferably the granule, tablet or capsule is administered to the patient or subject 2 times per day.

In one embodiment the enriched alcohol or water or a mixture thereof is concentrated and dried. Preferred concentration and drying steps include filtration, distillation, evaporation (including heat and or pressure evaporation), freeze drying and spray drying.

The composition disclosed herein also provide a dietary, nutraceutical or oral pharmaceutical composition consisting of Chaihu, Yujin, Mudanpi, Zicao, Baiwei, Baishao and Wuweizi or extracts thereof.

The composition disclosed herein may be formulated for administration in any chosen dosage form. Thus, for oral administration a dietary (a food, food additive or food supplement for example), nutraceutical or pharmaceutical composition useful herein may be formulated by a skilled worker according to known formulation techniques.

For example, foods, food additives or food supplements comprising a composition described herein for use according to the invention include any edible consumer product which is able to carry plant extracts. Examples of suitable edible consumer products include confectionary products, reconstituted fruit products, snack bars, muesli bars, spreads, dips, diary products including yoghurts and cheeses, drinks including dairy and non-dairy based drinks, milk powders, sports supplements including dairy and non-dairy based sports supplements, food additives such as protein sprinkles and dietary supplement products including daily supplement tablets. Suitable nutraceutical compositions useful herein may be provided in similar forms.

A suitable pharmaceutical composition may be formulated with appropriate pharmaceutically acceptable excipients, diluents or carriers selected with regard to the intended dosage form and standard pharmaceutical formulation practice. A dosage form useful herein can be administered orally as a powder, liquid, tablet or capsule. Suitable dosage forms may contain additional agents as required, including emulsifying, antioxidant, flavouring or colouring agents. Dosage forms useful herein may be adapted for immediate, delayed, modified, sustained, pulsed or controlled release of the active components. A preferred composition described herein for use herein comprises Chaihu, Yujin, Mudanpi, Zicao, Baiwei or extracts thereof and one or both of Baishao and Wuweizi or extracts thereof. Preferably the composition comprises Chaihu, Yujin, Mudanpi, Zicao, Baiwei, Baishao and Wuweizi or extracts thereof.

A composition as described herein may also be administered by parenteral routes including but not limited to subcutaneous, intravenous, intraperitoneal, intramuscular and intratumoural administration. Preferably the composition is administered via oral administration. Those skilled in the art will be able to prepare suitable formulations for parenteral administration without undue experimentation. The composition may be used alone or in combination with one or more other therapeutic agents. When used in combination with another therapeutic agent the administration of the two agents may be separate, simultaneous or sequential. Simultaneous administration includes the administration of a single dosage form that comprises both agents and the administration of the two agents in separate dosage forms at substantially the same time. Sequential administration includes the administration of the two agents according to different schedules, preferably so that there is an overlap in the periods during which the two agents are provided. Suitable agents with which the compositions of the invention can be co-administered include chemotherapeutic and immunotherapeutic agents, and other suitable agents known in the art. Such agents are preferably administered parenterally, preferably by intravenous, subcutaneous, intramuscular, intraperitoneal, intramedullar, epidural, intradermal, transdermal (topical), transmucosal, intra-articular, and intrapleural, as well as oral, inhalation, and rectal administration. Additionally, it is contemplated that a composition in accordance with the invention may be formulated with additional active ingredients which may be of benefit to a subject in particular instances. For example, therapeutic agents that target the same or different facets of the disease process may be used. As will be appreciated, the dose of the composition administered, the period of administration, and the general administration regime may differ between subjects depending on such variables as the severity of symptoms of a subject, the type of disorder to be treated, the mode of administration chosen, and the age, sex and/or general health of a subject. It should be appreciated that administration may include a single daily dose or administration of a number of discrete divided doses as may be appropriate. It should be understood that a person of ordinary skill in the art will be able without undue experimentation, having regard to that skill and this disclosure, to determine an effective dosage regime (including daily dose and timing of administration) for a given condition.

In one embodiment a composition of the invention is administered simultaneously or sequentially with chemotherapeutic, anti-cancer, immunotherapeutic, hormonotherapeutic or endocrine therapeutic agents given for gynaecological cancers such as tamoxifen, taxanes, taxol, paclitaxel (taxol), doxetaxel (taxotere), cyclophosphamide (docettaxel), alkylating agents (mitotic inhibitors), doxorubicin (paditaxel, adriamycin), antibiotics, mitoxantrone (novantrone), vinorelbine (navelbine), vinca alakaloids (mitotic inhibitors), fluoro-uracile, pyrimidine analogs (antimetabolites), capecitabine (xeloda), methotrexate, folic acid analog (anti-metabolites); hormonal therapies such as tamoxifen (Nolvadex)—antiestrogens, toremifene (fareston)—antiestrogens, letrozole Femara)—aromatase inhibitors, anastrazole (Arimidex)—aromatase inhibitors, exemestane (Aromasin)—aromatase inhibitors, goserelin (Zoladex)—LHRH inhibitors, leuprolide (Leurpon)—LHRH inhibitors, fulvestrant (Faslodex); surgery (eg, lumpectomy or mastectomy for breast cancers); radiation; monoclonal antibody therapy (trastuzumab (herceptin).

In one embodiment a composition of the invention comprises one or more hormonotherapeutic agents such as tamoxifen (TAM).

Various aspects of the invention will now be illustrated in non-limiting ways by reference to the following examples.

### EXPERIMENTAL

### Example 1

The following herbs in a dried form were weighed out and placed in a container: 10 g of Chaihu, 10 g of Yujin, 15 g of Mudanpi, 20 g of Zicao, 15 g of Baiwei, 15 g of Baishao, 15 g of Wuweizi.

Approximately 500 ml of water was added per 100 g of dried herbs. The herbs were then left in the water for approximately 30 minutes. Following this a decoction of the herbs was prepared by simmering the mixture of water and herbs for approximately 20 to 30 minutes.

An aliquot (approximately 100 ml) of the enriched water was removed from the container and collected. Further 100 ml aliquots were prepared by filling the container with an amount of cold water equal to the amount of enriched water that was removed.

The 100 ml aliquots of enriched water were then stored for use.

### Example 2

Thirty female breast cancer patients who were using tamoxifen (TAM) were administered 100 ml of a composition of the invention twice per day. Each patient had been diagnosed pathologically. The average age of the patients was 48 (38-64). Of all these patients, 21 (70%) patients were both ER+/PR+ (estrogen receptor and progesterone receptor positive); 5 patients (16%) were ER+/PR− (estrogen receptor positive and progesterone receptor negative) and 4 patients (13%) were ER−/PR+. The average time from starting use of TAM to occurrence of hot flashes symptoms was 2.4 months (1-5 months). According to the method of Kupperman S et al. (“Comparative clinical evaluation of estrogenic preparation by the menopausal and amenorrheal indices.” Journal of Clinical Endocrinology 1953; 13:688-703) the symptoms were divided into mild grade (times/day) and serious grade (>5 times/day). Symptom improvement was observed after three weeks of taking the TCM and the results are shown in Table 1. The grade of serious symptoms in 16 (53%) patients became mild. The grade of the mild symptoms in 14 (46%) patients almost disappeared.

TABLE 1

| | | | Symptoms | Symptoms |
|---|---|---|---|---|
| | | | Before | After |
| Patient | Age | ER/PR | (flashes/day) | (flashes/day) |
| 1 | 41 | −/+ | >5 | <5 |
| 2 | 38 | +/+ | >5 | <5 |
| 3 | 41 | +/+ | >5 | <5 |
| 4 | 47 | +/+ | ≦5 | 0 |
| 5 | 61 | +/+ | ≦5 | 0 |
| 6 | 64 | +/+ | ≦5 | 0 |
| 7 | 58 | +/+ | >5 | <5 |
| 8 | 64 | +/+ | ≦5 | 0 |
| 9 | 52 | +/+ | >5 | <5 |
| 10 | 62 | +/+ | ≦5 | 0 |
| 11 | 64 | +/− | ≦5 | 0 |
| 12 | 57 | +/− | ≦5 | 0 |
| 13 | 60 | +/+ | ≦5 | 0 |
| 14 | 52 | +/+ | >5 | <5 |
| 15 | 48 | −/+ | >5 | <5 |
| 16 | 47 | −/+ | ≦5 | 0 |
| 17 | 64 | +/− | ≦5 | 0 |
| 18 | 62 | +/+ | ≦5 | 0 |
| 19 | 56 | +/+ | >5 | <5 |
| 20 | 47 | +/− | >5 | <5 |
| 21 | 51 | +/− | >5 | <5 |
| 22 | 63 | +/+ | ≦5 | 0 |
| 23 | 45 | +/+ | >5 | <5 |
| 24 | 43 | +/+ | >5 | <5 |
| 25 | 54 | +/+ | >5 | <5 |
| 26 | 44 | +/+ | >5 | <5 |
| 27 | 58 | +/+ | ≦5 | 0 |
| 28 | 40 | −/+ | >5 | <5 |
| 29 | 52 | +/+ | >5 | <5 |
| 30 | 41 | +/+ | ≦5 | 0 |

### Example 3

68 breast cancer patients suffering hot flash symptoms (caused by hormonotherapy) 3 times per day or more were given a composition of the invention or a placebo in a double blind randomized trial. Patients were orally administered 100 ml of a composition of the invention, 3 times per day for 3 weeks. 66 patients completed the study. Of these 66 patients, 33 received a composition of the invention while 33 received a placebo. Patients recorded the number of hot flashes per day for 1 week before treatment began. This record was used as the baseline to appraise the improvements in occurrence and severity of hot flashes and in improvement in sleep. The method Kupperman referred to above was used to record the severity of hot flashes: ≦5 times/day=mild; >5 times/day=severe. The distribution of the two groups was similar in terms of age, body condition & severity of hot flashes.

### Improvement in Hot Flashes Symptoms

In the placebo group (33), 69.7% (23/33) experienced no change or effect and the remaining 30.3% (10/33) experienced slight relief from symptoms.

In the treatment group (33), 42.4% (14/33) experienced no change or effect, 42.4% (14/33) experienced some form of relief and for the remaining 15.2% (5/33), the hot flashes symptoms disappeared.

### Grouping*Kupperman Score Improvement Cross Tabulation

| | | | Kupperman score | | | |
|---|---|---|---|---|---|---|
| | | | Gradual Improvement | | | |
| | | | 0 | 1 | 2 | Total |
| Grouping | control | Count | 23 | 10 | | 33 |
| | | % | 69.7% | 30.3% | | 100.0% |
| | | within | | | | |
| | | Grouping | | | | |
| | treatment | Count | 14 | 14 | 5 | 33 |
| | | % | 42.4% | 42.4% | 15.2% | 100.0% |
| | | within | | | | |
| | | Grouping | | | | |
| Total | | Count | 37 | 24 | 5 | 66 |
| | | % | 56.1% | 36.4% | 7.6% | 100.0% |
| | | within | | | | |
| | | Grouping | | | | |

### Symmetric Measures

| | | Asymp. | | |
|---|---|---|---|---|
| | | Std. | Approx. | Approx. |
| | Value | Error(a) | T(b) | Sig. |
| Ordinal by | .553 | .170 | 2.738 | .006 |
| Gamma | | | | |
| Ordinal | | | | |
| N of Valid Cases | 66 | | | |

(a)Not assuming the null hypothesis.
(b)Using the asymptotic standard error assuming the null hypothesis.

There is an obvious improvement in the treatment group (P=0.006).

### Improvement in Quality of Sleep

In the placebo group, 60.6% (20) experienced no change while 39.4% (13) sees an improvement in sleeping quality.

In the treatment group, 36.4% (12/33) experienced no change while the other 63.6% (21/33) experienced an improvement in their sleep quality. These results are statistically significant (P=0.049).

### Case Processing Summary

| | Cases | | | | | |
|---|---|---|---|---|---|---|
| | Valid | | Missing | | Total | |
| | N | Percent | N | Percent | N | Percent |
| Grouping * | 66 | 97.1% | 2 | 2.9% | 68 | 100.0% |
| Curative | | | | | | |
| Effects of | | | | | | |
| Sleep | | | | | | |

### Grouping*Curative Effects of Sleep Cross Tabulation

| | | | Curative Effects of | | |
|---|---|---|---|---|---|
| | | | Sleep | | |
| | | | 0 | 1 | Total |
| Grouping | control | Count | 20 | 13 | 33 |
| | | % | 60.6% | 39.4% | 100.0% |
| | | within | | | |
| | | Grouping | | | |
| | treatment | Count | 12 | 21 | 33 |
| | | % | 36.4% | 63.6% | 100.0% |
| | | within | | | |
| | | Grouping | | | |
| Total | | Count | 32 | 34 | 66 |
| | | % | 48.5% | 51.5% | 100.0% |
| | | within | | | |
| | | Grouping | | | |

No patients reported any side effects. Heart, liver, kidney and blood tests were all normal.

The data above show oral administration of a composition of the invention improves the symptoms of hot flashes and improves the quality of sleep for breast cancer patients.

### INDUSTRIAL APPLICATION

The compositions and methods of the invention may be used to treat or prevent climacteric symptoms such as hot flashes.

1. A composition comprising Wuweizi or an extract thereof, and Baishao or an extract thereof, in combination with at least one of Baiwei or an extract thereof, Zicao or an extract thereof, or Chaihu or an extract thereof.

2. A composition of claim 1 further comprising Mudanpi or an extract thereof.

3. A composition of claim 1 further comprising Yujin or an extract thereof.

4. A composition of claim 3 comprising:
(a) about 3% (dry weight) to about 12% (dry weight) of Chaihu or an extract thereof,
(b) about 3% (dry weight) to about 12% (dry weight) of Yujin or an extract thereof,
(c) about 5% (dry weight) to about 15% (dry weight) of Mudanpi or an extract thereof,
(d) about 12% (dry weight) to about 20% (dry weight) of Zicao or an extract thereof,
(e) about 5% (dry weight) to about 15% (dry weight) of Baiwei or an extract thereof,
(f) about 5% (dry weight) to about 15% (dry weight) of Baishao or an extract thereof, and
(g) about 5% (dry weight) to about 15% (dry weight) of Wuweizi or an extract thereof,
or multiples thereof yet retaining approximate proportionality.

5. A composition of claim 3 wherein said composition comprises about 10% (dry weight) of Chaihu, about 10% (dry weight) of Yujin, about 15% (dry weight) of Mudanpi, about 20% (dry weight) of Zicao, about 15% (dry weight) of Baiwei, about 15% (dry weight) of Baishao and about 15% (dry weight) of Wuweizi or multiples thereof yet retaining approximate proportionality.

6. A composition of claim 1 wherein the plant material is in a dried form.

7. A composition of claim 1 wherein said plant material is in a fresh form.

8. A composition of claim 1 wherein said composition further comprises one or more hormonotherapeutic agents.

9. A composition of claim 1 wherein said composition further comprises a pharmaceutically acceptable carrier.

10. A composition of claim 1 wherein said extract is substantially a water extract.

11. Method of treating climacteric symptoms, comprising administering a medicament comprising a composition comprising Wuweizi or an extract thereof, and Baishao or an extract thereof, in combination with at least one of Baiwei or an extract thereof, Zicao or an extract thereof, Mudanpi or an extract thereof, Yujin or an extract thereof, or Chaihu or an extract thereof to a patient in need of such treatment.

12. Method according to claim 11, wherein said patient is selected from the group consisting of: a natural pre-menopausal or menopausal woman; a patient suffering symptoms caused by drug-induced menopause or surgical-induced menopause wherein said drug-induced menopause is menopause caused by chemotherapeutic, anti-cancer, immunotherapeutic, hormonotherapeutic or endocrine therapeutic agents; a patient with a gynaecological cancer wherein said gynaecological cancer is breast, ovarian, endometrial, cervical or vaginal cancer; a patient with gynaecological disorders wherein said gynaecological disorder is endometriosis; a patient with hysterectomy; and a patient for which Hormone Replacement Therapy would increase the risk of gynaecological cancer onset or relapse, and wherein optionally, said climacteric symptoms of said patient comprise at least one of hot flashes and impaired sleep quality.

13. Method according to claim 11 comprising administering said medicament simultaneously or sequentially with one or more hormonotherapeutic agents.

14. Method according to claim 11, comprising administering said medicament as a single composition.

15. A process for preparing a composition comprising the step of obtaining a herbal extract from a solvent containing Wuweizi or an extract thereof, and Baishao or an extract thereof, in combination with at least one of Baiwei or an extract thereof, Zicao or an extract thereof, or Chaihu or an extract thereof.

16. A process of claim 15 wherein said solvent further contains Mudanpi or an extract thereof.

17. A process of claim 15 wherein said solvent further contains Yujin or an extract thereof.

18. A process of claim 15, comprising adding about 50 to about 1000 ml of solvent per about 100 g of plant material.

19. A composition comprising Chaihu or an extract thereof, Yujin or an extract thereof, Mudanpi or an extract thereof, Zicao or an extract thereof, Baiwei or an extract thereof, Baishao or an extract thereof and Wuweizi or an extract thereof.
